(19) United States
(12) Patent Application Publication
(10) Pub. No.: US 2014/0120168 A1
(43) Pub. Date: May 1, 2014

(54) METASTABLE SILVER NANOPARTICLE COMPOSITES

(71) Applicant: nanoComposix, Inc., San Diego, CA (US)

(72) Inventors: STEVEN J. OLDENBURG, San Diego, CA (US); RICHARD K. BALDWIN, San Diego, CA (US)

(21) Appl. No.: 14/063,904

(22) Filed: Oct. 25, 2013

Related U.S. Application Data
(60) Provisional application No. 61/795,866, filed on Oct. 26, 2012.

(51) Int. Cl. A61K 9/14; A61K 33/38

(52) U.S. Cl. CPC A61K 9/146; A61K 33/38
USPC 424/497; 424/618; 424/490

(57) ABSTRACT

Embodiments of the present invention relate to a metastable silver nanoparticle composite, a process for its manufacture, and its use as a source for silver ions. In various embodiments, the composite comprises, consists essentially of, or consists of metastable silver nanoparticles that change shape when exposed to moisture, a stability modulant that controls the rate of the shape change, and a substrate to support the silver nanoparticles and the modulant.

### INCORPORATION BY REFERENCE TO ANY PRIORITY APPLICATIONS

This application claims the benefit of priority from U.S. Provisional Application 61/795,866, filed on Oct. 26, 2012, which is incorporated by reference in its entirety. Any and all applications for which a foreign or domestic priority claim is identified in the Application Data Sheet as filed with the present application are hereby incorporated by reference under 37 CFR 1.57.

### BACKGROUND

1. Field of the Invention

Various embodiments of the present invention relate to a metastable silver nanoparticle composite, a process for its manufacture, and its use as a source for silver ions. In various embodiments, the composite comprises, consists essentially of, or consists of metastable silver nanoparticles that change shape when exposed to moisture, a stability modulant that controls the rate of the shape change, and a substrate to support the silver nanoparticles and the modulant.

2. Description of the Related Art

Silver is a well-known broad spectrum antimicrobial. Both ionic and nanoparticle forms of silver have been integrated into a number of biomedical devices to increase the efficacy of treatment. For example, Nucryst Pharmaceuticals has developed Acticoat (e.g. U.S. Pat. No. 6,989,156) which contains nanocrystalline silver that has enhanced solubility and sustained release of silver ions. Other silver dressings include Silvercell, aquacell and MeipexAG.

All of the known silver dressing have ion release profiles that are a function of their local environment.

### SUMMARY

In one embodiment, the control over the ion release profile is an important factor in the efficacy of treatment. There is a need for a more general class of composites where the time release of silver ions is modulated by the physical and chemical properties of the composite. Provided herein are several embodiments of a composite comprising metastable silver nanoparticles and a stability modulant having antimicrobial activity for use in the prevention of bacterial, fungal and yeast growth.

Provided herein in one embodiment is a composite comprising a metastable silver nanoparticle, a stability modulant and a substrate, and where the silver nanoparticles undergo a change in shape when the composite is exposed to moisture.

In one embodiment, the silver nanoparticles in the composite are coated with a stability modulant that modifies the silver nanoparticle's ion release rate in a dry environment or a moist environment.

In one embodiment, the composite contains a coating that can is released when the composite is exposed to moisture, where the released coating modifies the silver nanoparticle's ion release rate in a moist environment.

In one embodiment, the composite contains a stability modulant particle that is bound to the substrate and can dissolve in a moist environment over time to modify the silver nanoparticle's ion release rate in a moist environment. In some embodiments, stability modulants can either be etchants which include but are not limited to oxidants or protectants which include but are not limited to barriers to prevent silver ion release, reductants or both. In one embodiment, etchants increase the rate or amount of silver ion release while protectants slow or decrease the amount of silver ion release.

In one embodiment, the color of the composite indicates the concentration and the shape of the silver nanoparticles bound to the substrate.

In one embodiment, the composite is used to treat wounds.

In one embodiment, a composite comprises a metastable silver nanoparticle and a stability modulant, where the silver nanoparticle undergoes a change in shape when the composite is exposed to moisture. In various embodiments, the composite further comprises a substrate. In various embodiments, the silver nanoparticles are nanoplates, nanopyramids, nanocubes, nanorods, or nanowires. In one embodiment, the silver nanoparticles are not spheres and undergo a reduction in aspect ratio when exposed to moisture. In one embodiment, the silver nanoparticles undergo a reduction in aspect ratio when exposed to water.

In one embodiment, the nanoparticles are faceted and the vertices between their crystal faces undergo an increase in radius of curvature on exposure to moisture. In one embodiment, the stability modulant is a surface coating on the silver nanoparticles. In various embodiments, the surface coating is an oxide, a polymer, organic ligand, thiol, stimulus responsive polymer, polyvinylpyrollidone, silica, polystyrene, tannic acid, polyvinylalcohol, polystyrene or polyacetylene. In one embodiment, the stability modulant is a chemical that is dried onto the substrate. In one embodiment, the chemical is an oxidant. In various embodiments, the chemical is a borate salt, a bicarbonate salt, a carboxylic acid salt, sodium borate, sodium bicarbonate, sodium ascorbate, chlorine salts, primary amines or secondary amines. In one embodiment, the stability modulant is a mixture of etchants and protectants.

In one embodiment, the stability modulant is a population of particles. In one embodiment, the particles release chlorine salts or chemicals with primary or secondary amines over a period of time greater than 30 minutes (e.g., 45 minutes, 50 minutes, 60 minutes, 2 hours or more).

In one embodiment, the composite further comprises a protectant on the surface of the particle and a reductant bound to the substrate. In one embodiment, the substrate is a porous network of fibers. In various embodiments, the substrate is a sheet, sock, sleeve, wrap, shirt, pant, mesh, cloth, sponge, paper, filter, medical implant, medical dressing or bandage. In one embodiment, the silver nanoparticles are primarily crystalline.

In one embodiment, at least 50% of the silver nanoparticle surface area is a silver ion lattice in the {111} crystal orientation. In one embodiment, the composite releases silver ions over a period of time greater than 30 minutes. In one embodiment, the silver nanoparticles are physisorbed, covalently bonded, or electrostatically bound to the substrate.

### BRIEF DESCRIPTION OF THE DRAWINGS

Further objects, features and advantages of the invention will become apparent from the following detailed description taken in conjunction with the accompanying figures showing illustrative embodiments of the invention, in which the following is a description of the drawings. The drawings are examples, and should not be used to limit the embodiments. Moreover, recitation of embodiments having stated features is not intended to exclude other embodiments having additional features or other embodiments incorporating different combinations of the stated features. Further, features in one embodiment (such as in one figure) may be combined with descriptions (and figures) of other embodiments.

FIG. 1A illustrates one embodiment of a cubic nanoplate that has a small radius of curvature.

FIG. 1B illustrates one embodiment of a cubic nanoplate with a larger radius of curvature.

FIG. 2A illustrates one embodiment of a generally plate shaped nanoparticle with a specific width and thickness.

FIG. 2B illustrates a one embodiment of a change of shape into another particle that has an increased thickness and a decreased width.

FIG. 3 illustrates the optical spectra of one embodiment of silver nanoplates that have different aspect ratios.

FIG. 4 shows a transmission electron microscopy (TEM) image of one embodiment of silver nanoplates after synthesis.

FIG. 5 shows a TEM image of one embodiment of silver nanoplates after five days.

FIG. 6 shows a chart that documents the optical shift associated with the shape change of silver nanoplates according to one embodiment of the invention.

FIG. 7A illustrates one embodiment of a composite that contains fibers and metastable silver particles.

FIG. 7B shows metastable silver particles that are plate shaped according to one embodiment of the invention.

FIG. 7C shows metastable silver particles that are plate shaped and coated with a stability modulant according to one embodiment of the invention.

FIG. 8A illustrates a one embodiment of a composite that contains fibers, metastable silver particles and a chemical stabilant.

FIG. 8B illustrates the chemical coating component that is applied to the fiber and nanoparticles to form the composite according to one embodiment of the invention.

FIG. 9 illustrates a composite that contains fibers, metastable silver particles and particles that release a stability modulant over time according to one embodiment of the invention.

FIG. 10A illustrates a bandage that contains metastable silver particles attached to a woven mesh according to one embodiment of the invention.

FIG. 10B illustrates a close-up view of the metastable silver particles attached to a woven mesh according to one embodiment of the invention.

### DETAILED DESCRIPTION OF THE PREFERRED EMBODIMENT

Several embodiments of this invention include a composite that when exposed to moisture releases silver ions. In various embodiments, the composite comprises, consists essentially of, or consists of metastable silver nanoparticles, a stability modulant and a substrate. Metastable silver nanoparticles can be any shape. In certain embodiments the metastable silver nanoparticles have a non-spherical shape. In various embodiments, shapes that may be metastable include spheres, plates, discs, rods, wires, triangular, pyramidal, bipyrimidal, cubes, and other crystalline faceted shapes. In one embodiment a substantial portion of the metastable silver nanoparticles have a plate shape and are referred to as nanoplates. In one embodiment, silver nanoplates are characterized by lengths along the three principle axes wherein the axial length of two of the principle axes is at least two times greater than the axial length of the shortest principle axis and the shortest principal axial length is less than about 500 nm (e.g., 400 nm, 300 nm, 250 nm, 100 nm or less) and greater than zero (e.g., 0.5 nm, 1 nm, 5 nm, or more) or any range therein. Silver nanoplates have a variety of different cross sectional shapes including circular, triangular, or shapes that have any number of discrete edges. In one embodiment the nanoplates have less than 20, 15, 10, 8, 6, 5, or 4 edges (e.g., 3 edges, 2, edges, 1 edges). In one embodiment the nanoplates have more than 2, 3, 4, or 5 edges (e.g., 7, 8, 12, 17 or more edges). In some embodiments the silver nanoplates have sharp corners and in other embodiments the corners are rounded. In some embodiments of silver nanoplates, there are a variety of different cross sectional shapes within the same sample. In other embodiments of silver nanoplate solutions greater than 5%, 10%, 20%, 30%, 40%, 50%, 60%, 70%, 80%, or 90% of the number of particles in solution are silver nanoplates with the other particles having different shapes including, but not limited to, spherical, cubic, and/or irregular. In some embodiments the nanoplates have one or two flat sides. In one embodiment the nanoplates are pyramidal. In some embodiments the particles are primarily crystalline. In some embodiments at least 10%, 20%, 50%, 75% or 90% (e.g., 15%, 55%, 95%) of the silver nanoparticle surface is in the {111} crystal orientation.

In one embodiment, the nanoparticles have a rod shape. Silver rods are characterized by lengths along the three principle axes wherein the axial length of one of the principle axes is at least two times greater than the axial length of the other two principle axis and the shortest principal axial length is less than about 500 nm (e.g., 400 nm, 300 nm, 250 nm, 100 nm or less) and greater than zero (e.g., 0.5 nm, 1 nm, 5 nm, or more) or any range therein.

In one embodiment, the nanoparticles have a cubic shape. Cubes have six flat generally equal faces. In some embodiments the faces of the cubes meet at a sharp edge. In other embodiments the edges where two faces meet are rounded. In other embodiments the corners of the cubes are rounded. The radius of curvature of the edges or corners is defined to be the radius of a circle that best matches the outer dimensions of a cross sectional cut through the vertex, edge or corner of the cube.

In one embodiment, the nanoparticles have multiple facets or sides. In some embodiments a side has a surface roughness less than 10%. The edges or vertices of the faces can have different radii of curvature. In one embodiment a nanoparticle is pyramidal in shape where the figure has a polygonal base and triangular faces that meet at a common point.

In one embodiment the shape of the particles is a bipyramid that consists of two pyramids with a common polygonal base.

In one embodiment, the metastable silver nanoparticles are generally spherical. The silver nanoparticles change shape by decreasing in size over time in the presence of stability modifiers.

In one embodiment, the aspect ratio of a nanoparticle is referred to as the ratio between the longest principal axis and the shortest principal axis. In one embodiment the average aspect ratio of the metastable nanoparticles is greater than about 1.5, 2, 3, 4, 5, 7, 10, 20, 30, or 50 (e.g., 15, 25, 60, 100 or more). In one embodiment the average aspect ratio of the metastable nanoparticles is between 1.5 and 25, 2 and 25, 1.5 and 50, 2 and 50, 3 and 25, or 3 and 50 (e.g, 10 and 15, 12 and 17, 35 and 45, etc.). In various embodiments, the nanoparticle has edge lengths less than about 500 nm, 250 nm, 200 nm, 150 nm, 100 nm, 80 nm, 60 nm or 50 nm. In various embodiments, the nanoparticle has edge lengths greater than about 5 nm, 10 nm, 20 nm, 30 nm, 50 nm or 100 nm. In one embodiment the nanoparticle has a thickness (third principle axis) that is less than about 500 nm, 300 nm, 200 nm, 100 nm, 80 nm, 60 nm, 50 nm, 40 nm, 30 nm, 20 nm, or 10 nm.

In an embodiment, the silver nanoparticles are metastable with respect to their shape. Metastable nanoparticles have a fixed size and shape under one set of environmental conditions but then undergo a size or shape change under another set of environmental conditions. In various embodiments, examples of shape changes include a reduction in aspect ratio, a change in the local radius of curvature at the vertex between two crystal faces, a transformation to a more spherical shape, the deposition of metal ions onto one or more surfaces of the nanoparticle, or a change in the surface roughness of the particle. In an embodiment, the silver nanoparticles have a high aspect ratio or highly faceted shape and when exposed to moisture silver ions from one portion of the nanoparticle are released into solution and redeposit on another portion of the particle. In one embodiment the silver nanoparticles are plate shaped and the primary dissociation of the silver ions occurs at the edges of the particle and is deposited primarily onto the faces of the nanoparticle which reduces the aspect ratio of the particle. In an embodiment, the silver nanoparticles have a rod or wire shape and in a moist environment, silver ions are released from the ends of the rods or wires and deposit onto the long axis surface of the particles resulting in a reduced aspect ratio.

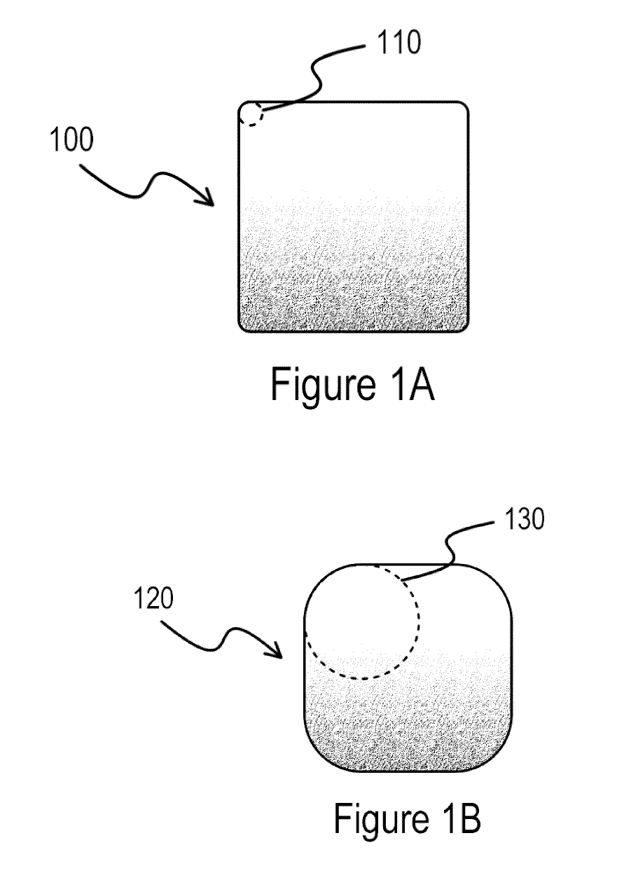

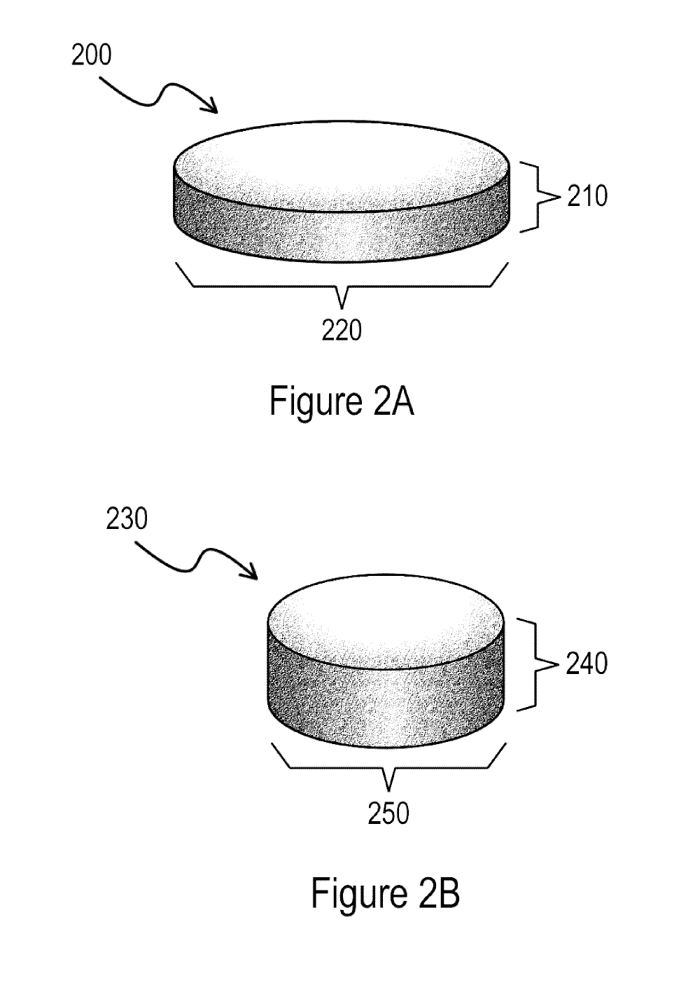

FIG. 1A illustrates one embodiment of a generally cubic plate silver nanoparticle 100 that has a radius of curvature at its corners defined by the circle 110. Under certain environmental conditions a shape change can occur and in some embodiments this can result in an increased radius of curvature at the corners of the nanoparticle. FIG. 1B illustrates one embodiment of a generally cubic plate silver nanoparticle 120 that has an increased radius of curvature 130 when compared to the radius of curvature 110. FIG. 2A illustrates one embodiment of a generally plate shaped nanoparticle 200 with a thickness 210 and a width 220. In an embodiment, under certain environmental conditions the shape of the plate shaped nanoparticle 200 can change shape into another particle 230, illustrated in FIG. 2B that has an increased thickness 240 and a decreased width 250.

In an embodiment the degree to which the particles are metastable is controlled by the particular crystal facets that the nanoparticle expresses. Different crystal facets have different degrees of lability of silver ion associated with them. By controlling the facets that are expressed on the nanoparticle, the off rate of silver ions from the silver nanoparticle surface can be controlled.

In an embodiment the silver nanoparticles can have a pyramidal shape and an oxidation process generating silver ions that leads to an increase in the radius of curvature of the vertex between one or more crystal faces.

In an embodiment the silver nanoparticles can have a cubic shape and on exposure to moisture undergo an oxidation process releasing silver ions, leading to an increase in the radius of curvature of the vertex between one or more crystal faces.

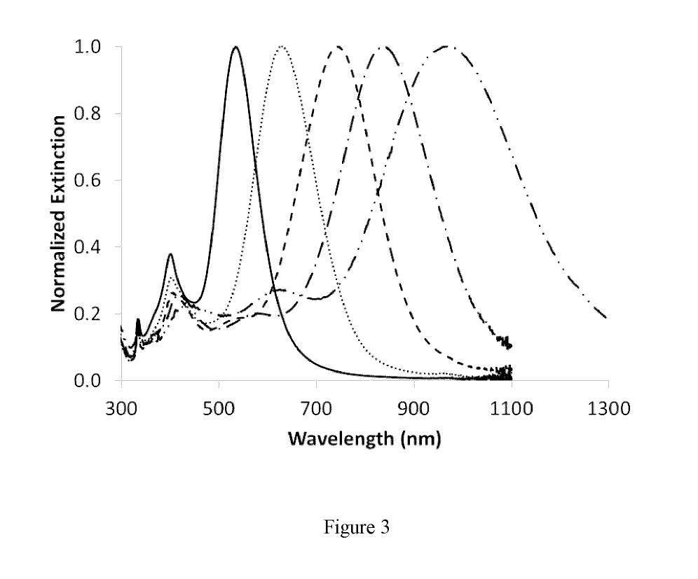

In an embodiment, the change in the shape of silver nanoparticle modifies the optical properties of the silver nanoparticles. Silver nanoparticles can support surface plasmon modes and referred to as a plasmon resonant particles. FIG. 3 illustrates the optical spectra of one embodiment of silver nanoplates that have different aspect ratios. Each of these particles in solution has a different color that is discernible by the eye. In one embodiment, the shape of the nanoparticles will change due to ion dissolution from the surface of the nanoparticle where the silver ion dissolution rate is approximately the same at all points on the surface of the nanoparticle. This results in the size of the particle being reduced. In one embodiment, the ion dissolution rate from the surface of the nanoparticle is not the same at all points on the surface. For example, the ion release rate from the edges of a plate shape nanoparticle may be greater than the ion release rate from the surface of the particle. In this case, the shape change of the particle is due to a change in the aspect ratio of the particle. In one embodiment, the silver ions that are released from the surface either stay in solution or complex with other chemicals or surfaces. In one embodiment, the silver ions that are released from the surface can rebind to the same silver nanoparticle or to other silver nanoparticles in the composite. The rebinding of the silver ions to the silver nanoparticles can be uniform on all silver surfaces or can preferentially bind to one or more faces of the silver nanoparticles. In one embodiment, the silver ion release rate and the silver ion deposition rate is a function of the size of the particle. For example, the silver ion release rate can be greater for smaller particles than for larger particles. In one embodiment, the free silver ions in solution form new silver nanoparticles. When new silver nanoparticles are formed they are generally spherical and the shape distribution of the nanoparticles on the substrate or in solution can be different than the original shape distribution.

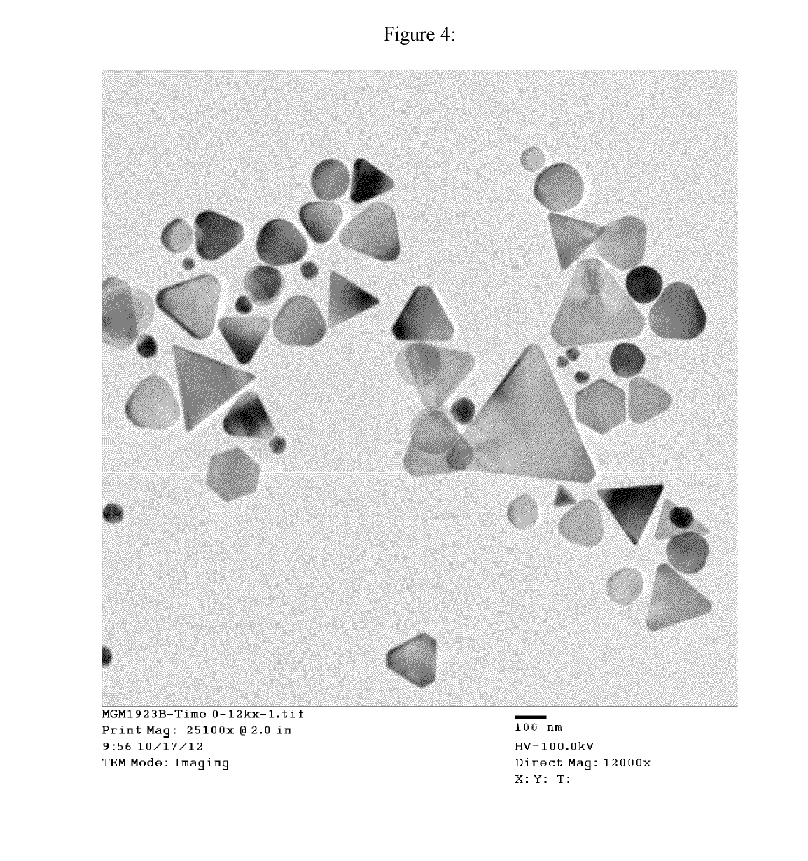

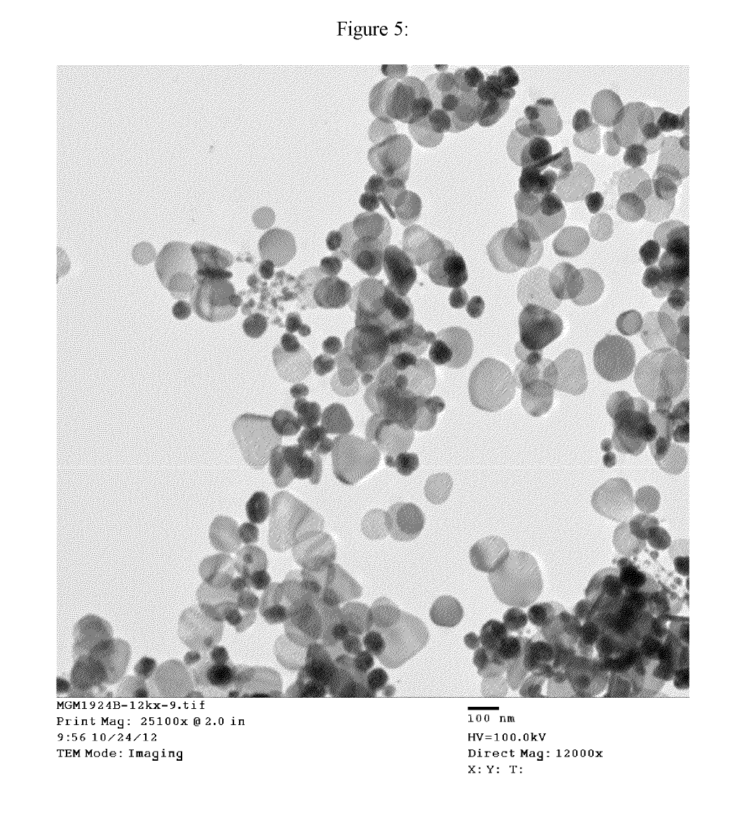

FIG. 4 illustrates transmission electron microscopy (TEM) images of some embodiments of silver nanoplates immediately after synthesis. FIG. 5 illustrates a TEM image of one embodiment of silver nanoparticles that were stored in an open container for 5 days.

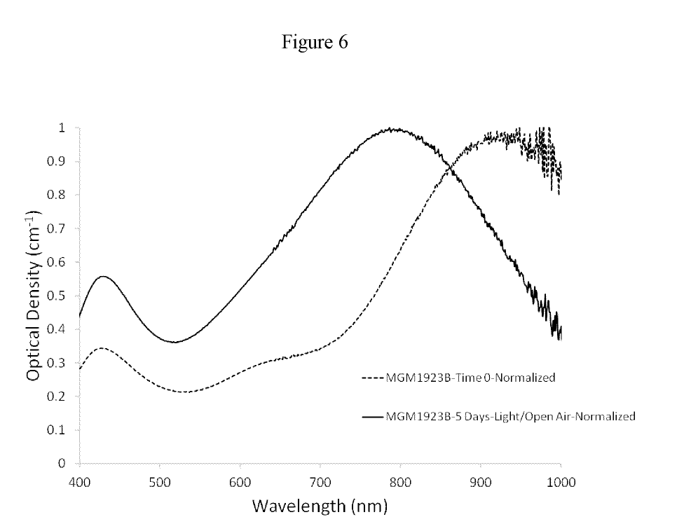

FIG. 6 shows the UV Visible spectrum of the one embodiment of particles that have changed shape over time. The ratio of spheres to disks to triangles was 18:28:53 for the TEM sample in FIG. 4 (time 0) and 38:47:16 for the TEM sample in FIG. 5 (time 5 days). The average diameter of the spheres, disks, and triangles was 55 nm, 130 nm, and 170 nm, respectively for the TEM sample in FIG. 4 (time 0). The average diameter of the spheres, disks, and triangles was 61 nm, 116 nm, and 137 nm, respectively in FIG. 5 (time 5 days). This data demonstrates that both the distribution of shapes and the sizes is changing with time. The peak extinction wavelength was initially 930 nm. Five days later, the peak extinction wavelength was 790 nm. The shape change induced a peak extinction wavelength shift of 140 nm. In some embodiments, a peak wavelength shift of at least 5 nm, 10 nm, 20 nm, or 50 nm constitutes a perceptible shift in the color of the particles.

In one embodiment, the visible color shift that is associated with the change in the shape of the metastable particles provides information on the state of the silver nanoparticles. The color change of the silver nanoparticles is associated with the shape of the particle which in turn is a function of the silver ion release rate and the silver ion deposition rate on the silver nanoparticles. The end user of the composite can utilize both the color intensity (measuring how much is loaded onto the composite) and the color wavelength (the current shape of the particle) to determine the state of the silver nanoparticles in the composite. In one embodiment, the color can be used to determine whether the composite is still efficacious for wound treatment. In one embodiment, the color can be used to determine whether or not a washing step removed or altered the silver nanoparticles in the composite.

In an embodiment, the degree to which the particles are metastable is controlled by the environment. In some embodiments the medium surrounding the silver nanoparticles is a gas which can include gases such as air or an inert atmosphere. In some embodiments the environment is a full or partial vacuum. In an embodiment, the metastable nanoparticles can undergo a chemical change associated with the long term storage in the gas environment. This change can include the oxidation of the silver or the binding of aerosolized molecular species to the surface of the silver including molecules that contain amines or mercapto components. In one embodiment the medium is moist. A moist environment is defined to be wet, slightly wet, damp, or humid. In the case where the moist environment is a liquid, the liquid can be a pure liquid or any combination of liquids. In a preferred embodiment, the liquid media consists of a substantial portion of water and is referred to as an aqueous medium. The liquid media can also contain a percentage of chemical or biological solids. In one embodiment the aqueous medium is a biological fluid such as a wound exudate, blood, or serum. In some embodiments, the moist environment creates a liquid layer near the surface of the silver nanoparticles. In this embodiment, silver ions can diffuse from the surface of the nanoparticles into solution. In an embodiment, the Ag⁰ of the metal nanoparticles is oxidized into solubleAg⁺¹ ions. Free silver ions in solution can remain in solution, bind to another entity in contact with the solution, or be reduced back to Ag⁰ on the surface of the silver nanoparticles or somewhere else.

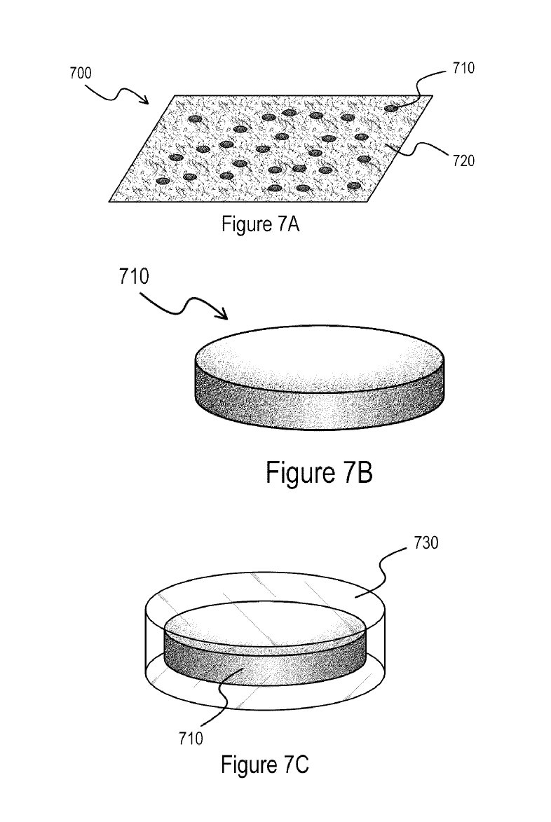

In an embodiment, the proposed composite includes a stability modulant. A stability modulant is any material that affects the stability of the metastable nanoparticles. In one embodiment the stability modulant is a coating on the nanoparticle that increases the stability of the metastable nanoparticles. FIG. 7A illustrates a composite 700 that consists of silver nanoparticles 710 and a substrate 720. In one embodiment, the silver nanoparticles are coated with an encapsulant 730 illustrated in FIG. 7C. Nanoparticles coated with a stabilant can retain their shape for days, weeks, months or years in either or both wet or dry environments. The stabilant can be a chemical or biological agent that is physibsorbed to the surface, molecularly bound to the surface through specific interactions (e.g. thiol or amine), or encapsulate the surface (i.e. a metal oxide or metalloid oxide shell). In various embodiments, examples of chemical agents that can be bound to the surface of the silver nanoparticles include citric acid, polysulphonates, vinyl polymers, alkane thiols, carbohydrates, ethylene oxides, phenols, and carbohydrates. In some embodiments the silver nanoparticles are coated with poly(sodium) styrene sulfonate, polyvinyl alcohol, polyvinyl pyrrolidone, tannic acid, dextran, and polyethylene glycol (PEG) including PEG molecules which contain one or more chemical groups (e.g. amine, thiol, acrylate, alkyne, maleimide, silane, salts (e.g. sodium borate or sodium bicarbonate), azide, hydroxyl, lipid, disulfide, fluorescent molecule, or biomolecule moieties). In various embodiments, specific biomolecules of interest include proteins, peptides, and oligonucleotides, including biotin, bovine serum albumin, streptavidin, neutravidin, wheat germ agglutinin, naturally occurring and synthetic oligonucleotides and peptides, including synthetic oligonucleotides which have one or more chemical functionalities (e.g. amine, thiol, dithiol, acrylic phosphoramidite, azide, digoxigenin, alkynes, or biomolecule moieties). Specific encapsulating chemical agents of interest include metal oxide shells such as SiO₂ and TiO₂. Stabilizing agents may be added prior to the formation of silver nanoparticles, during the formation of silver nanoparticles, or after the formation of silver nanoparticles. The thickness of the coating can be a monolayer or sub-monolayer or a shell that fully or partially encapsulates the nanoparticle. The thickness of the shell can range from 1 nm to 100 nm. In some embodiments the shell is porous (e.g. silica).

In an embodiment, the metastable silver nanoparticles are combined with one or more stability modulants into a paste, cream, or liquid. In one embodiment the metastable silver nanoparticles are coated with a protectant. In one embodiment, the suspension medium contains an etchant. In one embodiment, a combination of etchants and protectants are combined with the silver nanoparticles into the suspension medium.

In one embodiment, the stability modulant can affect the binding strength of the silver nanoparticle to the substrate. For example, proteases or other biological processes in a wound bed could accelerate the release rate of the silver nanoparticle from the substrate into the local environment. In one embodiment, the stability modulant is an acid, solvent, or other biological or chemical entity that can interact with the binding forces adhering a silver nanoparticle to the substrate.

In various embodiments, metallic silver nanoparticles, on exposure to air and water, can undergo oxidation to generate silver ions. The extent and the nature of this oxidation depends on the environment of the silver and the shape of the silver nanoparticles. In one embodiment, the nanoparticles are shelled with a layer that modulates access of the oxidizing species to the surface which controls the rate at which the silver ionizes. In one embodiment, the stability modulant protects the silver nanoparticles from thiols. In an embodiment the use of a layer of oxide such as silica, or a layer of polymer such as polystyrene on the surface of the silver nanoparticles, can control the rate of generation of silver ions from the surface.

In one embodiment, the use of a reductant on the surface of the silver nanoparticles can reduce the oxidation of the silver on the silver nanoparticle. In one embodiment, the reductant on the surface of the silver is fully or partially removed from the surface when the silver nanoparticles is exposed to moisture. In one embodiment the reductant is in the form of an ascorbate, citrate or other organic or inorganic reductant and is closely associated with the surface of the silver metal nanoparticles until dissolved away with moisture. In one embodiment the reductant stays in close proximity to the silver and reduces the off rate of silver ions from the surface regardless of the moisture conditions.

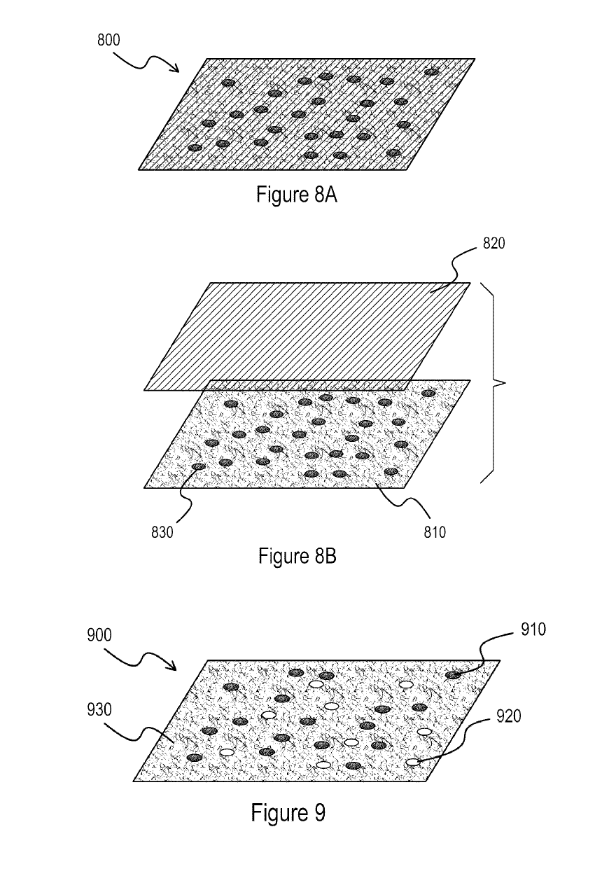

In one embodiment, there is a stability modulant in the composite that is a material that accelerates the dissolution of the metastable silver nanoparticles. In one embodiment, the stabilant modulant is added to the composite as a coating. FIG. 8A illustrates an embodiment of a composite 800 that consists of a substrate, silver nanoparticles and a coating. FIG. 8B illustrates the components of the composite 800. The coating 820 is applied to the substrate 810 which contains silver nanoparticles 830. The stabilant modulant is dissolved when the composite comes in contact with moisture which affects the properties of the liquid that is contact with the composite (the environment). In some embodiments the stabilant modulants either raises or lowers the pH of the environment, contains molecules that can displace or dissolve surface coatings or shells on the silver particles, contains amines, contains thiols, contains oxidants, contains salts, contains etchants, or contains halides. In some embodiments, the stabilant modulant coating rapidly dissolves. In other embodiments, the stabilant modulant coating is mixed with other compounds that slow the release of the stabilant modulant allowing the modulant to be released over a period of hours, days, weeks, or months. In one embodiment the stabilant modulant is a population of particles that are bound to the substrate. FIG. 9 illustrates a composite 900 that consists of silver nanoparticles 910 and stability modulant particles 920 that are attached to a substrate 930. In one embodiment the particles can dissolve with time to release stabilant modulant molecules that accelerate the dissolution of the silver nanoparticles. The particles can be made from a single stabilant modulant, a combination of stabilant modulants, or can include other chemicals and the stabilant modulant. The other chemicals present in the particle can include slow release compounds such as PLGA.

In an embodiment, an oxidant can be employed to increase the silver ion off rate from the particles. This can include any species likely to oxidize silver and the oxidant can stem from the environment, the composite it is placed in or can be a part of the composite itself. Example oxidants include but are not limited to amines, thiols, other metal salts or oxidizing organic species.

In an embodiment, a combination of oxidant and reductant can be employed in the composite to modulate the rate and amount of silver ion dissolution. In a particular embodiment the reductant is associated with the surface of the silver nanoparticles, preventing generation of the ions until it is desired to do so. In one embodiment the oxidant is spatially displaced from the surface of the silver nanoparticles and it water soluble. On exposure to moisture, the reductant is displaced from the surface of the silver nanoparticles and the surface is exposed to an oxidant which has diffused to the surface consequently increasing the rate of dissolution of the silver nanoparticles on exposure to moisture.

In some embodiments, the composite includes a coating that increases the stability of the silver nanoparticles during dry storage and additional stability modulants in the composite that accelerate the dissolution of the silver nanoparticles when exposed to moisture. In some embodiments, the composite is stable for long periods of time when not in use and stored in a wide variety of temperature and humidity environments while retaining the ability to release silver ions when in a moist environment. In one embodiment the coating on the particles is a porous shell (e.g. silica). In other embodiments, the coating on the particle increases the binding strength to the substrate.

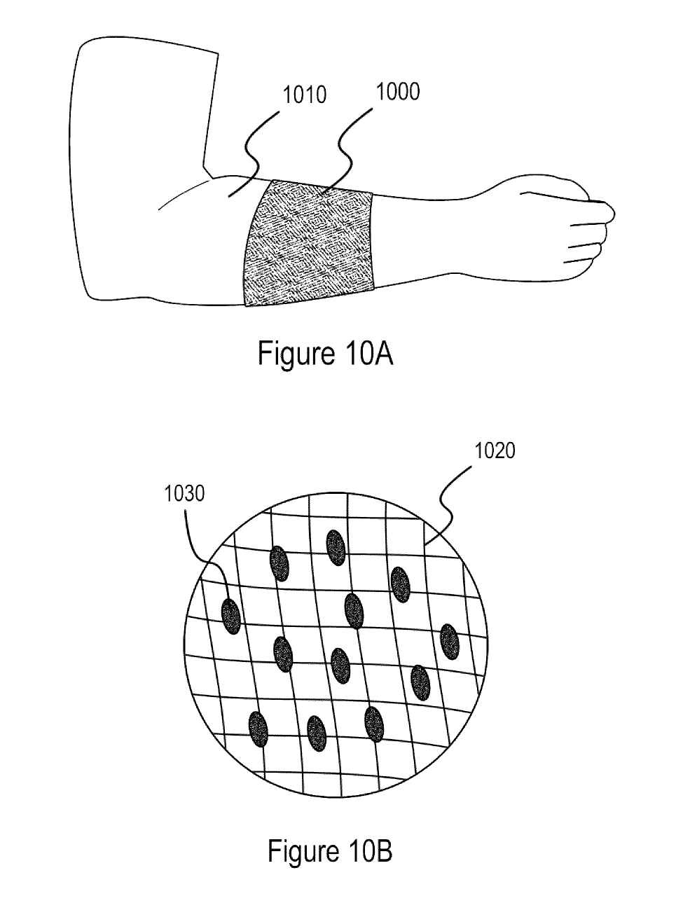

In one embodiment of the invention, the metastable silver nanoparticles are associated with a substrate. Examples of substrates include non-woven fibers, woven fibers, natural fibers, fibers from animals (e.g. wool, silk), plant (e.g. cotton, flax, jute), mineral fibers (e.g. glass fiber), synthetic fibers (nylon, polyester, acrylic), cloth, mesh, bandages, socks, wraps, other articles of clothing, sponges, high porosity substrates, particles with diameters greater than 1 micron, beads, hair, skin, paper, absorbant polymers, foam, wood, cork, slides, roughened surfaces, biocompatible substrates, filters, or medical implants. FIG. 10A illustrates a bandage 1000 that is applied to an arm (1010). FIG. 10B shows a close-up of the structure of the bandage 1000. The substrate is a cloth of woven or otherwise combined fiber 1020 that has silver nanoparticles 1030 bound to the surface of the fiber.

In one embodiment, the high optical density solutions of silver nanoparticles at a concentration of at least 1 mg/mL, 10 mg/mL, 100 mg/mL (e.g., 1 to 10, 3 to 30, 5 to 50, 10 to 20, 5 to 50, 3 to 50, 1 to 100 mg/mL, 10 to 100, 20 to 100, 30 to 100 mg/mL) are incubated with the substrate. In one embodiment, the high optical density solutions of silver nanoparticles at a concentration of at least 1 mg/mL, 10 mg/mL, or 100 mg/mL are incubated with the substrate. In one embodiment the silver nanoparticles are prepared at an optical density of at least 10, 100, 300, 500, 1000, or 2000 cm⁻¹ before incubating with the substrate. In one embodiment the substrate is chemically treated to increase the binding of the silver nanoparticles to the substrate. For example, the substrate could be functionalized with a molecule that yielded a positively or negatively charged surface. In one embodiment, the pH of the incubating solution is selected in order to optimize binding. In one embodiment, the silver nanoparticles cover at least 5%, 10%, 20%, 30%, 50% or 75% of the substrate. In one embodiment, other solvents or chemicals are added to the incubation solution. In one embodiment a biological linker (e.g. antibodies, peptides, DNA) is used to bind the high optical density silver nanoparticles to the surface of the substrate. In one embodiment the substrate is chemically modified to have a higher affinity to the silver nanoparticles. In a particular embodiment a protein based substrate in which dithiol bridges are present is reduced, generating free thiols that can bind to the surface of the silver nanoparticle. In one embodiment, the incubation is for less than 1 minute, 5 minutes, 20 minutes, 60 minutes, or 120 minutes. In one embodiment the silver nanoparticles are physisorbed, covalently bounded, or electrostatically bound to the substrate. In one embodiment, the faces of the high aspect ratio particles that have the largest surface area preferential bind to the substrate. In one embodiment, silver nanoparticles with a high aspect ratio shape bind with more force to the substrate than silver nanoparticles with a lower aspect ratio.

In one embodiment, the composite does not release silver ions in the dry state and is only activated to release silver ions in the presence of moisture. The moisture can be from a high humidity environment, dipping or spraying the composite with a water based compound, or from the composite being in contact with a moist surface. Examples of moist surfaces include wounds such as burns, lacerations, ulcers, non-healing wounds, cuts, gun shot wounds, and injuries due to explosive fragmentation. Other types of surfaces that the composite can be applied to include clothing, foot wear, socks, wraps, compression bandages, porous surfaces (e.g. porous surfaces on furniture and equipment), medical devices, and other surfaces that need to be sterile.

In one embodiment, the metastable silver nanoparticles and the stability modulant have been optimized to release silver ions over an extended period of time. In some embodiments, the local concentration of silver ions in and around the composite when exposed to a moist environment for the first time is at least 5 ppb, 10 ppb, 20 ppb, 40 ppb 100 ppb, 300 ppb, 500 ppb, 1000 ppb, 2 ppm, 5 ppm, 10 ppm 40 ppm, or 100 ppm or more. In some embodiments the silver ion release rate is at least 20%, 30%, 50%, or 70% of the initial silver ion release rate value after 12 hours. In some embodiments, the silver on the composite is mostly retained after a wash step. In some embodiments, at least 30%, 50%, 80%, 90% or 95% of the initial silver is retained after a wash cycle of the composite.

Shaped silver nanoparticles are fabricated using methods known in the literature. For example, silver nanoplates can be fabricated using photoconversion (Jin et al. 2001; Jin et al. 2003), pH controlled photoconversion (Xue 2007), thermal growth (Hao et al. 2004; Hao 2002; He 2008; Metraux 2005), templated growth (Hao et al. 2004; Hao 2002), seed mediated growth (Aherne 2008; Chen; Carroll 2003; Chen; Carroll 2002, 2004; Chen et al. 2002; He 2008; Le Guevel 2009; Xiong et al. 2007), or alternative methods.
Aherne, D. L., D. M.; Gara, M.; Kelly, J. M., 2008: Optical Properties and Growth Aspects of Silver Nanoprisms Produced by Highly Reproducible and Rapid Synthesis at Room Temperature. Advanced Materials, 18, 2005-2016.
Chen, S., and D. L. Carroll, 2003: Controlling 2-dimensional growth of silver nanoplates. Self-Assembled Nanostructured Materials Symposium (Mater. Res. Soc. Symposium Proceedings Vol. 775), 343-348|xiii+394.
Chen, S. H., and D. L. Carroll, 2002: Synthesis and characterization of truncated triangular silver nanoplates. Nano Letters, 2, 1003-1007.
Chen, S. H., and D. L. Carroll, 2004: Silver nanoplates: Size control in two dimensions and formation mechanisms. Journal of Physical Chemistry B, 108, 5500-5506.
Chen, S. H., Z. Y. Fan, and D. L. Carroll, 2002: Silver nanodisks: Synthesis, characterization, and self-assembly. Journal of Physical Chemistry B, 106, 10777-10781.
Hao, E., G. C. Schatz, and J. T. Hupp, 2004: Synthesis and optical properties of anisotropic metal nanoparticles. Journal of Fluorescence, 14, 331-341.
Hao, E. K., K. L.; Hupp, J. T.; Schatz, G. C., 2002: Synthesis of Silver Nanodisks using Polystyrene Mesospheres as Templates. J Am Chem Soc, 124, 15182-15183.
He, X. Z., X.; Chen, Y.; Feng, J., 2008: The evidence for synthesis of truncated silver nanoplates in the presence of CTAB. Materials Characterization, 59, 380-384.
Jin, R., Y. Cao, C. A. Mirkin, K. L. Kelly, G. C. Schatz, and J. G. Zheng, 2001: Photoinduced Conversion of Silver Nanospheres to Nanoprisms. Science, 294, 1901-1903.
Jin, R., Y. C. Cao, E. Hao, G. S. Metraux, G. C. Schatz, and C. A. Mirkin, 2003: Controlling anisotropic nanoparticle growth through plasmon excitation. Nature, 425, 487.
Le Guevel, X. W., F. Y.; Stranik, O.; Nooney, R.; Gubala, V.; McDonagh, C.; MacCraith, B. D., 2009: Synthesis, Stabilization, and Functionalization of Silver Nanoplates for Biosensor Applications. J Phys Chem C, 113, 16380-16386.
Metraux, G. S. M., C. A; 2005: Rapid Thermal Synthesis of Silver Nanoprisms with Chemically Tailorable Thickness. Advanced Materials, 17, 412-415.
Xiong, Y. J., A. R. Siekkinen, J. G. Wang, Y. D. Yin, M. J. Kim, and Y.
N. Xia, 2007: Synthesis of silver nanoplates at high yields by slowing down the polyol reduction of silver nitrate with polyacrylamide. Journal of Materials Chemistry, 17, 2600-2602.
Xue, C. M., C. A., 2007: pH-Switchable Silver Nanoprism Growth Pathways. Angew Chem Int Ed, 46, 2036-2038.

Each of the references listed above is incorporated by reference in its entirety.

Alternative methods include methods in which the silver nanoparticles are formed from a solution comprising a shape stabilizing agent or agents and a silver source, and in which chemical agents, biological agents, electromagnetic radiation, or heat are used to reduce the silver source. Synthesis methods for other shapes and sizes of silver nanoparticles are reported in the scientific literature.

Other embodiments of the invention will be apparent to those skilled in the art from consideration of the specification and practice of the invention disclosed herein. It is intended that the specification and examples be considered as disclosing certain embodiments of the invention only, with a true scope and spirit of the invention being indicated by the following claims.

The subject matter described herein may be embodied in other specific forms without departing from the spirit or essential characteristics thereof. The foregoing embodiments are therefore to be considered in all respects illustrative rather than limiting. While embodiments are susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited.

The methods disclosed herein include certain actions taken by a practitioner; however, they can also include any third-party instruction of those actions, either expressly or by implication. For example, actions such as “application to a target region of skin tissue” include “instructing the application to a target region of skin tissue.”

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” or “substantially” include the recited numbers. For example, “about 3 mm” includes “3 mm.” The terms “approximately”, “about” and/or “substantially” as used herein represent an amount or characteristic close to the stated amount or characteristic that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount or characteristic.

### EXAMPLES

The description of specific examples below are intended for purposes of illustration only and are not intended to limit the scope of the invention disclosed herein.

### Example 1

### Silver Nanoplates

Silver nanoplates were synthesized using silver seeds prepared through the reduction of silver nitrate with sodium borohydride in the presence of sodium citrate tribasic and poly sodium styrene sulfonate under aqueous conditions. Silver seed preparation: 21.3 mL of an aqueous 2.5 mM sodium citrate tribasic solution was allowed to mix under magnetic stirring. 1 mL of a 2 g/L poly styrene sodium sulfonate (PSSS) solution was then prepared in a separate beaker. 21.3 mL of a 0.5 mM silver nitrate solution was then prepared by dissolving the salt in water. Once the above solutions have been prepared, 1.33 mL of a 0.5 mM sodium borohydride solution should be prepared using cold water. The borohydride and PSSS solutions were then added to the beaker containing the citrate and allowed to mix. The silver nitrate solution was then pumped into the citrate solution using a peristaltic pump at a rate of 100 mL/min. This seed solution was then allowed to stir overnight at room temperature. Silver nanoplate preparation: Silver nanoplates were prepared by mixing 1530 mL Milli-Q water with 35 mL of a 10 mM ascorbic acid solution. Once the solution sufficiently mixed, the silver seed (made 24 h prior) was added to the beaker. 353 mL of a 2 mM silver nitrate solution was then pumped into the beaker at a rate of 100 mL/min. Following the completion of the silver nitrate, the solution was allowed to mix at room temperature for at least two hours to allow the reaction to go to completion.

### Example 2

### Silica Shelling Silver Nanoplates

A silica shell was grown on the surface of 800 nm resonant (˜75 nm diameter polyvinylpyrolidone (PVP) capped silver nanoplates. 600 mL of a solution of 800 nm resonant PVP40T capped silver nanoplates at a concentration of 1 mg/mL was added to 3.5 L of reagent grade ethanol and 270 mL Milli-Q water under constant stirring. 4.3 mL of dilute aminopropyl triethoxysilane (215 uL APTES in 4.085 mL isopropanol) was then added to the solution, followed immediately by the addition of 44 mL of 30% ammonium hydroxide.

After 15 minutes of incubation, 31 mL of dilute tetraethylorthosilicate (1.55 mL TEOS in 29.45 mL isopropanol) was added to the solution. The solution was then left to stir overnight. The nanoplates were then centrifuged on an Ultra centrifuge at 17000 rcf for 15 min and reconstituted in milli-Q water each time and repeated twice. The shell thickness was controlled by the amount of TEOS added.

### Example #3

### Binding to a Substrate

10 mL of silver nanoplates prepared at a concentration of 1 mg/mL were incubated with a 5 g coupon from a commercially available chamois (Detailer's Choice). The fluid was completely absorbed by the chamois and allowed to air dry to produce a darkly colored substrate.

### Example 4

### Addition of a Stability Modifier

10 mL of silver nanoplates prepared at a concentration of 1 mg/mL were incubated with a 5 g coupon from a commercially available chamois (Detailer's Choice). The fluid was completely absorbed by the chamois and allowed to air dry to produce a darkly colored substrate. The dried coupon was incubated with 3 mL of a 1M solution of NaCl and heat dried to produce a substrate with a stability modifier dried into the sample.

### Example 5

### Silver Ion Release Rates

The silver ion concentration of 1 mg/mL 10 nm silver nanoparticles was measured to be 3 ppb within 12 hours of synthesis and increased to 22 ppb after 4 days. The silver ion concentration of silver nanoplates in a sodium borate buffer was 9 ppb after 2 days. The silver ion concentration of silver nanoplates in a water solution was 1160 ppb after 1 day.

What is claimed is:

1. A composite comprising a metastable silver nanoparticle and a stability modulant where the silver nanoparticle undergoes a change in shape when the composite is exposed to moisture.

2. The composite of claim 1 further comprising a substrate.

3. The composite of claim 1 where the silver nanoparticles are nanoplates, nanopyramids, nanocubes, nanorods, or nanowires.

4. The composite of claim 1 where the silver nanoparticles are not spheres and undergo a reduction in aspect ratio when exposed to moisture.

5. The composite in claim 3 where the silver nanoparticles undergo a reduction in aspect ratio when exposed to water.

6. The composite in claim 1 where the nanoparticles are faceted and the vertices between their crystal faces undergo an increase in radius of curvature on exposure to moisture.

7. The composite of claim 1 where the stability modulant is a surface coating on the silver nanoparticles.

8. The composite of claim 7 where the surface coating is any one selected from the group consisting of an oxide, a polymer, organic ligand, thiol, stimulus responsive polymer, polyvinylpyrollidone, silica, polystyrene, tannic acid, polyvinylalcohol, polystyrene and polyacetylene.

9. The composite of claim 2 where the stability modulant is a chemical that is dried onto the substrate.

10. The composite of claim 9 where the chemical is an oxidant.

11. The composite of claim 9 where the chemical is any one selected from the group consisting of a borate salt, a bicarbonate salt, a carboxylic acid salt, sodium borate, sodium bicarbonate, sodium ascorbate, chlorine salts, primary amines and secondary amines.

12. The composite of claim 9 where the stability modulant is a mixture of etchants and protectants.

13. The composite of claim 1 where the stability modulant is a population of particles.

14. The composite of claim 13 where the particles release chlorine salts or chemicals with primary or secondary amines over a period of time greater than 30 minutes.

15. The composite of claim 2 where there is a protectant on the surface of the particle and a reductant bound to the substrate.

16. The composite of claim 2 where the substrate is a porous network of fibers, a sheet, sock, sleeve, wrap, shirt, pant, mesh, cloth, sponge, paper, filter, medical implant, medical dressing or bandage.

17. The composite of claim 1 where the silver nanoparticles are primarily crystalline.

18. The composite of claim 1 where at least 50% of the silver nanoparticle surface area is a silver ion lattice in the {111} crystal orientation.

19. The composite of claim 1 where the composite releases silver ions over a period of time greater than 30 minutes.

20. The composite of claim 2 where the silver nanoparticles are physisorbed, covalently bonded, or electrostatically bound to the substrate.
